(19) United States
(12) Patent Application Publication
(10) Pub. No.: US 2026/0030967 A1
(43) Pub. Date: Jan. 29, 2026

(54) HAZARD DETECTOR WITH REPLACEABLE DETECTOR MODULE

(71) Applicant: RESIDEO LLC, Golden Valley, MN (US)

(72) Inventors: Travis Read, Little Canada, MN (US); Ryan Thorsen, Fergus Falls, MN (US); Pat Tessier, Maple Grove, MN (US)

(73) Assignee: RESIDEO LLC, Golden Valley, MN (US)

(21) Appl. No.: 19/144,072

(22) PCT Filed: Dec. 27, 2023
(86) PCT No.: PCT/US2023/085997

§ 371 Date: Jun. 27, 2025

Related U.S. Application Data
(60) Provisional application No. 63/477,678, filed on Dec. 29, 2022.

(51) Int. Cl. G08B 21/02; G01N 33/00

(52) U.S. Cl. CPC G08B 21/02; G01N 33/004; G01N 33/0063; G01N 33/007

(57) ABSTRACT

A hazard detector device can comprise a base that defines a module receptacle and is configured to be secured to a surface and comprise a detector module configured to insert into the module receptacle and secure to the base. The detector module can include a battery, a hazard sensor powered by the battery, a memory configured to store data about the hazard sensor, and communication circuitry in communication with the memory and configured to communicate with an external device. A detector module can be replaced with another detector module without replacing the base.

### RELATED APPLICATIONS

This Application claims the benefit of U.S. Provisional Patent Application No. 63/477,678 filed Dec. 29, 2022, the contents of which are incorporated herein by reference.

### TECHNICAL FIELD

This disclosure generally relates to hazard detectors.

### BACKGROUND

Traditional residential hazard detectors (e.g., smoke detectors) comprise an entire unit having an integrated sensor part and integrated power, such as a battery, and/or an external power hookup, such as to a house's main power. In general, hazard detectors require replacement as components within the hazard detector can become unreliable over time. To replace a hazard detector, one needs to buy and install a new unit which can require electrically wiring the hazard detector to external power such as a house's mains power. Wiring to a house's mains power can be dangerous if not done properly. Further, it can be time consuming in places with many hazard detectors to replace them.

### SUMMARY

This disclosure in general describes a hazard detector device having a base and a hazard detector module that is easily replaceable. In particular, embodiments disclosed herein enable a user to easily replace hazard sensors without requiring any wiring or specific knowledge in replacing hazard sensors. Further, embodiments disclosed herein provide a reuseable base that can be used with many hazard sensors over its lifetime.

In a first aspect, a hazard detector device includes a base and a detector module. The base can define a module receptacle. The base can be configured to be secured to a surface. The detector module can be configured to insert into the module receptacle and secure to the base. The detector module can include a battery. The detector module can include a hazard sensor. The hazard sensor can be powered by the battery. The detector module can include a memory. The memory can be configured to store data about the hazard sensor. The detector module can include communication circuitry. The communication circuitry can be in communication with the memory. The communication circuitry can be configured to communicate with an external device.

The hazard detector device can include a variety of features. In some examples, the hazard sensor can include a smoke sensor and/or a carbon monoxide detector. The base can provide primary power to the detector module via one or more electrical contacts. The battery can provide backup power to the detector module.

In some examples, the base can include one or more of a temperature sensor, a humidity sensor, a motion sensor, a pressure sensor, or a light sensor. The detector module can be configured to receive one or more measurements from the one or more of the temperature sensor, the humidity sensor, the motion sensor, the pressure sensor, or the light sensor and use the one or more measurements with one or more measurements from the hazard sensor to determine if a hazard is present.

The base and the detector module can communicate electrically with each other. In some examples, the base can include one or more base electrical contacts. In some examples, the detector module can include one or more detector module electrical contacts. In some examples, the one or more detector module electrical contacts is in electrical communication with the one or more base electrical contacts when the detector module is inserted into the base. In some examples, the one or more base electrical contacts can include concentric circles of electrical contacts. In some examples, the one or more detector module electrical contacts can include spring-loaded electrical contacts.

In some examples, interaction between the base and the detector module can begin when the detector module is pressed into the base. In some examples, the base can include a switch. In some examples, the detector module can activate the switch of the base when the detector module is pressed into the base. When the detector module is pressed into the base, the detector may be configured to perform a self-test to assess operation of the detector module. The detector module may be configured to communicate a result of the self-test to the external device via the communication circuitry.

In some examples, the base can include an alarm (e.g., audio and/or visual) configured to produce an alert when activated by the detector module during a hazard event. In some such examples, when the detector module is fully inserted into the module receptacle, the alarm may be activated. The alarm may be configured to deactivate upon the detector module activating the switch of the base.

In some examples, the memory may be configured to store one or more items of information. The memory can be configured to store an installation date of the detector module when the detector module is first inserted into the base. The memory can be configured to store a manufacturing date of the detector module. The memory can be configured to store an expiration date of the detector module. In some examples, the memory can be configured to store a hazard sensor type. In some examples, the memory can be configured to store a battery type.

In some examples, structural features can facilitate the detector module inserting into the module receptacle. In some examples, the base can include a protrusion that extends at least partially into the module receptacle. In some examples, the detector module, when inserted into the module receptacle, can frictionally engage the protrusion to secure to the base. In some examples, the module receptacle and the detector module can be generally cylindrical. In some examples, the detector module can include a ridge. The ridge can be configured to contact the protrusion when the detector module is fully inserted into the module receptacle.

In some examples, the detector module can include a detecting portion. The detecting portion can be located outside the module receptacle when the detector module is secured to the base. The detecting portion can be configured to allow gases, heat, and particulates to reach the hazard sensor.

In some examples, the detector module can define a threaded hole. The threaded hole can face the base when the detector module is inserted into the module receptacle. The threaded hole can be configured to accept a fastener to secure the detector module to the base.

In a second aspect, a method can include securing a base to a surface (e.g., a ceiling) and mounting a hazard detector module to the base. The base can define a module receptacle. The base can include a protrusion proximate an outer edge of the module receptacle. The method can include inserting a hazard detector module into the module receptacle. A portion of the hazard detector module can frictionally engage the protrusion of the base. The hazard detector module can include a battery. The hazard detector module can include a hazard sensor (e.g., a smoke sensor). The hazard sensor can be powered by the battery. The hazard detector module can include a memory. The memory can be configured to store data about the hazard sensor. The hazard detector module can include communication circuitry. The communication circuitry may be in communication with the memory. The communication circuitry may be configured to communicate with an external device.

In a third aspect, a method can include removing a first detector module from a base and installing a second detector module into the base. The base can be secured to a surface. Removing the first detector module can include pulling the first detector module from a module receptacle of the base and overcoming frictional engagement therebetween. The first detector module can include a first battery. The first detector module can include a first hazard sensor. The first hazard sensor can be powered by the first battery. The first detector module can include a first memory. The first memory can be configured to store data about the first hazard sensor. The first detector module can include first communication circuitry. The first communication circuitry can be configured to communicate with an external device. Installing the second detector module into the base can include inserting the second detector module into the module receptacle of the base. Installing the second detector module into the base can include frictionally engaging the second detector module with the module receptacle.

In various examples, the second detector module can have the same, similar, or different attributes compared to the first detector module. In some examples, the second detector module can include a second battery. In some examples, the second detector module can include a second hazard sensor. The second hazard sensor can be powered by the second battery. In some examples, the second detector module can include a second memory. The second memory can be configured to store data about the second hazard sensor. In some examples, the second detector module can include second communication circuitry. The second communication circuitry can be configured to communicate with the external device. In some examples, the first battery can have capacity to power the first hazard sensor for between one year and three years. In some examples, the second battery can have capacity to power the second hazard sensor for more than three years. In some examples, the first hazard sensor can include a smoke sensor but not a carbon monoxide sensor. In some examples, the second hazard sensor can include a smoke sensor and a carbon monoxide sensor. In some examples, the first battery can include an alkaline battery. In some examples, the second battery can include a lithium battery. In some examples, the second detector module can include a replacement date on a front face of the second detector module, the front face facing away from the surface when the second detector module is installed. In some examples, the first detector module, the second detector module, and the module receptacle of the base can be round.

The details of one or more examples are set forth in the accompanying drawings and the description below. Other features, objects, and advantages will be apparent from the description and drawings, and from the claims.

### BRIEF DESCRIPTION OF DRAWINGS

The following drawings are illustrative of particular examples of the present invention and therefore do not limit the scope of the invention. The drawings are intended for use in conjunction with the explanations in the following detailed description wherein like reference characters denote like elements. Examples of the present invention will hereinafter be described in conjunction with the appended drawings.

FIG. 1 is a perspective view of an example hazard detector device including a base and detector module according to an aspect of the present disclosure.

FIG. 2A is a perspective view of an example detector module including a front face according to an aspect of the present disclosure.

FIG. 2B is a perspective view of an example detector module similar to that of FIG. 2A including a rear face according to an aspect of the present disclosure.

FIG. 3 is a perspective view of an example hazard detector device including a base and a detector module being inserted into the base according to an aspect of the present disclosure.

FIG. 4 is a flowchart of an example method of installing a hazard detector device according to an aspect of the present disclosure.

FIG. 5A and FIG. 5B are perspective views of alternate examples of detector modules according to aspects of the present disclosure.

FIG. 6 is a perspective view of an alternate example of a hazard detector device including a base and detector module according to an aspect of the present disclosure.

FIG. 7 is a partially transparent perspective view of a hazard detector device including a base and detector module with a security fastener according to an aspect of the present disclosure.

FIG. 8 is a flowchart of an example method of replacing a detector module of an example hazard detector device according to an aspect of the present disclosure.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

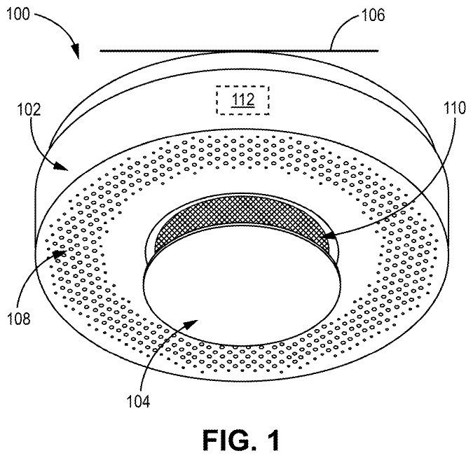

Starting with FIG. 1, FIG. 1 is a perspective view of an example hazard detector device 100 including a base 102 and detector module 104 according to an aspect of the present disclosure. The base 102 houses the detector module 104 with the base 102 able to provide alerting capabilities and the detector module 104 able to provide hazard detecting capabilities. The detector module 104 is configured to be removable from the base 102 such that a user can replace the detector module 104 if needed while still maintaining the base 102. The base 102 is in communication with the detector module 104 and, when the detector module 104 detects a hazard (e.g., smoke indicative of a fire), it can communicate with the base 102 such that the base 102 provides an alert (e.g., audio and/or visual alert). In some examples, the base 102 can be in electrical communication with the detector module 104 and can provide power to the detector module 104.

In the illustrated embodiment, the hazard detector device 100 is mounted to a surface 106 with a front face 108 of the base facing away from the surface 106. In some examples, the base 102 is mounted directly to the surface 106. However, in some examples, the base 102 can be indirectly mounted to the surface such as via a mounting plate. The base 102 includes a front face 108 which defines a series of holes that can allow sound to pass through. The base 102 also includes an audio alarm 112 which can comprise one or more speakers spread around the base 102 and can produce sound to pass through the series of holes. In some examples, the base includes a visual alarm in addition to or in lieu of the audio alarm 112. In some such examples, the base does not include the series of holes.

In FIG. 1, the detector module 104 is fully inserted into the base 102. However, even when fully inserted, the detector module 104 can include a detecting portion 110 which protrudes from the base 102. The detecting portion 110 generally allows particulates, gases, and/or heat to enter the detector module 104 so that they can be detected by the detector module 104.

As illustrated, the base 102 and the detector module 104 include text engraved thereon. The text can be engraved, printed, molded, or provided via any other method. The text on the base 102 and the detector module 104 can comprise any text. In some examples, the detector module 104 has a limited useful lifespan and as such, the text on the detector module 104 can indicate a replacement date on a front face of the detector module as shown in FIG. 1.

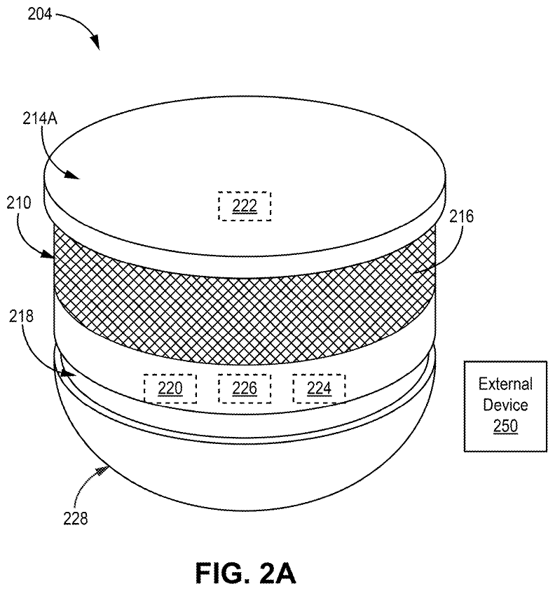

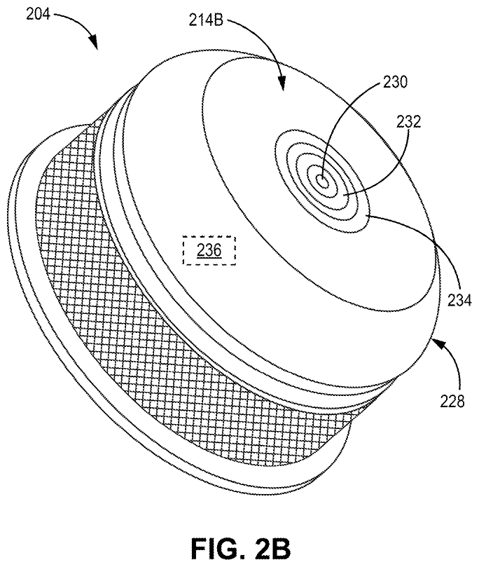

Moving to FIG. 2A and FIG. 2B, FIG. 2A is a perspective view of an example detector module 204 including a front face 214A according to an aspect of the present disclosure while FIG. 2B is a perspective view of an example detector module 204 very similar to that of FIG. 2A including a rear face 214B according to an aspect of the present disclosure. The detector module 204 includes a grill 216 and an indent 218 which are located on an exterior of the detector module 204. The detector module 204 also includes a battery 220, a hazard sensor 222, a memory 224, and communication circuitry 226 which are housed within the detector module 204.

Starting with the exterior, the detector module 204 is generally cylindrical with a tapered rear portion 228. The tapered rear portion 228 is a portion of the detector module 204 which can be inserted into a base (e.g., 102). The indent 218 of the detector module 204 is also a portion of the detector module which can be inserted into a base. As illustrated, the indent 218 surrounds the detector module 204. However, in some examples, the indent only surrounds a portion of the detector module. The indent 218 can be used as a securing point for one or more corresponding protrusions of the base and can prevent the detector module 204 from inserting further into the base.

The detector module 204 also includes the grill 216 which is part of a detecting portion 210 of the detector module 204. The grill 216 surrounds the detector module 204 and specifically surrounds the hazard sensor 222. The grill 216 can take many forms, but generally protects the hazard sensor 222 from external forces and/or external debris while still allowing gases (e.g., air), particulates (e.g., ash), and heat to reach the hazard sensor 222.

Moving to the interior, the detector module 204 includes the battery 220 and the hazard sensor 222. In the illustrated embodiment, the battery 220 provides power to the hazard sensor 222. However, the battery 220 can also provide power to other components within the detector such as the memory 224 and the communication circuitry 226. In some embodiments, the battery 220 only provides secondary or backup power to components of the detector module as is described elsewhere herein. The battery 220 can be any type of battery including but not limited to alkaline batteries and lithium batteries. The battery 220 is also not limited to a specific physical size nor specific capacity. For instance, in some examples, the battery 220 has a capacity to power the hazard sensor 222 for between one year and three years (e.g., 2 years) while in some examples, the battery 220 has a capacity to power the hazard sensor 222 for more than three years (e.g., 10 years). In some such examples, the capacity of the battery is based on the battery only powering the hazard sensor and with normal operation of both the battery and the hazard sensor. In some embodiments, the battery is a CR123 battery while in some embodiments, the battery is a coin cell battery. In some examples, the detector module includes more than one battery. A person having ordinary skill in the art will appreciate that any number of batteries of any physical size, type, capacity, voltage etc. can be used in the detector module 204 and that this disclosure is not limited to any one battery.

Continuing with the example of FIG. 2A, the hazard sensor 222 of the detector module 204 is configured to detect a hazard which can take many different forms. For instance, in some examples, the hazard detector can be a smoke sensor such as an ionization smoke sensor or a photoelectric smoke sensor. However, the hazard detector can include many different sensors including but not limited to gas sensors (e.g., carbon monoxide, oxygen, propane gas, natural gas, and methane), heat sensors, and particulate sensors. In operation, the hazard sensor can detect if a hazard is present and can provide an output indicating as such. In some examples, the output is relayed to a base (e.g., 102) which can provide an audio and/or visual alert indicating the presence of a detected hazard. Additionally or alternatively, in some examples, the output is relayed to one or more external devices 250 via communication circuitry (e.g., 226). In some embodiments, the hazard sensor 222 has a limited useful lifespan in which the hazard detector is considered reliable. In some such embodiments, the hazard detector has a limited useful lifespan of about 10 years.

Further in the embodiment of FIG. 2A, the detector module 204 includes a memory 224 and communication circuitry 226 which are in communication with each other. The memory 224 can store information (e.g., data) related to the detector module 204 while the communication circuitry 226 can communicate with an external device 250. In some examples, the communication circuitry 226 communicates wirelessly with the external device 250. In some examples, the communication circuitry 226 communicates with multiple external devices.

In the illustrated embodiment, the memory 224 can store information including but not limited to: an installation date of the detector module 204 when the detector module is first inserted into the base (e.g., 102), a manufacturing date of the detector module 204, an expiration date of the detector module 204, a hazard sensor 222 type, a battery 220 type, and a battery 220 capacity. In some embodiments, the memory 224 can store information related to a base (e.g., 102), an external device 250, and/or to the communication circuitry 226. One of ordinary skill in the art will appreciate the memory 224 is not limited to storing any specific information. Additionally, the memory can comprise any type of memory including volatile, non-volatile, programmable, and read-only memory.

In some examples, the communication circuitry 226 can communicate information related to the detector module 204 to the external device 250. In one such example, the communication circuitry 226 can communicate an expiration date of the hazard sensor 222 to the external device 250. In another such example, the communication circuitry 226 can communicate if the detector module 204 is properly inserted into the base and/or if the detector module 204, especially the hazard sensor 222, is working properly. In some examples, the communication circuitry 226 can indicate to an external device if the hazard sensor 222 is activated (e.g., indicating a hazard condition). For example, if the hazard sensor 222 is a smoke sensor and detects smoke, the communication circuitry 226 can indicate the hazard sensor detects smoke to the external device.

Now focusing on FIG. 2B, the detector module 204 includes electrical contacts 230, 232, 234 which can be referred to as detector module electrical contacts. As illustrated, electrical contacts 230, 232, 234 are concentric circles of conductive material which are generally flat or flush with the rear face 214B. A first electrical contact 230 is located proximate a center of the detector module, a second electrical contact 232 is located slightly further away from the center of the detector module, and a third electrical contact 234 is located even further away from the center of the detector module. In contrast to the illustrated example, in some examples, the detector module electrical contacts comprise electrical contacts which protrude from the detector module and can be spring-loaded and/or pogo-style electrical contacts. The electrical contacts 230, 232, 234 are electrically connected to circuitry 236 located within the detector module 204. The circuitry 236 can comprise a printed circuit board and can be used to receive/send power and/or data carried by the electrical contacts 230, 232, 234 to electronics within the detector module 204. For instance, in some examples, the electrical contacts 230, 232, 234 carry power to circuitry 236 to power the communication circuitry 226. However, in some examples, the electrical contacts 230, 232, 234 are electrically connected to electronics within the detector module 204 directly and circuitry 236 is not needed. In some embodiments, one or more electrical contacts carry data while one or more electrical contacts carry power. For instance, in one such embodiment, the first electrical contact 230 and the second electrical contact 232 carry power while the third electrical contact carries data. While three electrical contacts are illustrated, in some examples, one or more than one electrical contacts can be used.

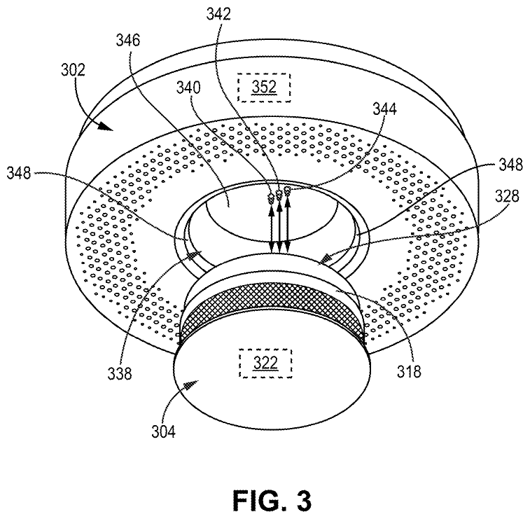

Moving to FIG. 3, FIG. 3 is an example hazard detector device including a base 302 and a detector module 304 being inserted into the base 302 according to an aspect of the present disclosure. As illustrated, the base 302 defines a module receptacle 338 and includes base electrical contacts 340, 342, 344, switch 346, protrusions 348, and an optional sensor 352. The detector module 304 includes a hazard sensor 322, tapered rear portion 328, and an indent 318. While not visible in the view of FIG. 3, the detector module 304 also includes detector module electrical contacts which comprise three concentric circles of conductive material. The detector module contacts in the embodiment of FIG. 3 are similar to those in the embodiment of FIG. 2 with a first detector module electrical contact located proximate a center of the detector module, a second detector module electrical contact located slightly further away from the center of the detector module, and a third detector module electrical contact located even further away from the center of the detector module.

Starting with the base 302, the base 302 includes the module receptacle 338. The module receptacle 338 is centered in the base 302, has a circular opening, a planar bottom on which the base electrical contacts 340, 342, 344 and the switch 346 are mounted, and is tapered between the circular opening and the planar bottom. The module receptacle 338 is circularly shaped and sized to accept the detector module 304. However, in some examples, the module receptacle 338 can be differently shaped and sized to fit a differently sized and shaped detector module. In such examples, the base electrical contacts can be located in a different area of the module receptacle.

Continuing with the base 302, the base can include the optional sensor 352. The optional sensor 352 can be any type of sensor including but not limited to: a temperature sensor, a humidity sensor, a motion sensor, a pressure sensor, or a light sensor. In some examples, more than one optional sensor is included in the base. The optional sensor 352 can interface with electronics in the base 302, including communication electronics, and can output measurements to the electronics in the base. In some examples, the optional sensor can be in communication with one or more of the base electrical contacts 340, 342, 344 and can output measurements via the one or more base electrical contacts 340, 342, 344.

Now referencing both the base 302 and the detector module 304, in the embodiment of FIG. 3, the detector module 304 is in the process of being inserted into the module receptacle 338 of the base 302. As the tapered rear portion 328 of the detector module 304 is inserted into the module receptacle 338, it can frictionally engage the protrusions 348. Eventually, the protrusions 348 in the module receptacle 338 can engage the indent 318 and can secure the detector module 304 in place relative to the base 302. In some examples, the detector module 304 does not include an indent, but includes a ridge which is secured via the protrusions. In some examples, the detector module 304 does not include an indent and the protrusions frictionally secure the detector module in place. In the illustrated example, the protrusions 348 comprise a compressible material that is compressed as the tapered rear portion 328 of the detector module 304 is inserted into the module receptacle 338, but which is uncompressed or less compressed when the protrusions engage the indent 318. This configuration enables the detector module 304 to be easily secured to the base 302 by simply inserting the detector module 304 into the module receptacle 338 without any further action required. For instance, the frictional fit of the detector module 304 to the base 302 does not require any rotation of the detector module relative to the base which may be difficult to perform if the base is secured to a hard to access surface. While the protrusions 348 are illustrated as being separate, in some examples, a single protrusion is used which surrounds the module receptacle 338. In some examples, one or more protrusions are used which can be of any length and be spaced apart any distance. In general, the protrusions are used to secure the detector module 304 to the base 302.

While the mechanism of FIG. 3 to secure the detector module to the base is advantageous, other mechanisms for securing the detector module to the base are contemplated. For instance, in some examples, the base includes an indent or ridge while the detector module includes protrusions which frictionally engage the base. In some examples, a rotatable locking mechanism is used to secure the detector module to the base. In some examples, fasteners and/or adhesives are used to secure the detector module to the base. A person having ordinary skill in the art will understand that this disclosure is not limited to the listed example securing mechanisms.

Continuing with the embodiment of FIG. 3, the base electrical contacts 340, 342, 344 are located on a planar portion of the base 302. A first base electrical contact 340 is located proximate a center of the module receptacle 338, a second base electrical contact 342 is located slightly further away from the center of the module receptacle 338, while a third base electrical contact is located even further away from the center of the module receptacle 338. In the illustrated example, the base electrical contacts 340, 342, 344 comprise spring-loaded electrical contacts which can be pogo-style electrical contacts. In some examples, though, the base electrical contacts comprise concentric circles of conductive material that is substantially flat (e.g., similar to the electrical contacts 230, 232, 234). The base electrical contacts 340, 342, 344 can be electrically connected to electronics within the base 302 including power electronics, communication electronics, sensor electronics, and light electronics. In some examples, the base is electrically connected to a power source (e.g., mains power of a house) and can transmit power through the base electrical contacts 340, 342, 344. Additionally or alternatively, the base is electrically connected to a communication source (e.g., home automation system) and can transmit data through the base electrical contacts 340, 342, 344. While three base electrical contacts are illustrated, in some examples, the base 302 includes one or more base electrical contacts.

Further in the embodiment of FIG. 3, when the detector module 304 is fully inserted into the module receptacle 338, the base electrical contacts 340, 342, 344 can contact the planar rear portion (e.g., rear face 214B) of the detector module 304 which comprises the detector module electrical contacts (e.g., those illustrated in FIG. 2B). As indicated by the arrows in the embodiment of FIG. 3, the base electrical contacts 340, 342, 344 each align with a different portion of the detector module 304 which corresponds to a detector module electrical contact. For instance, the first base electrical contact 340, which is located proximate a center of the module receptacle 338, is aligned with a first detector module electrical contact (e.g., 230 of FIG. 2). Further, the second base electrical contact 342, which is located slightly further from the center of the module receptacle 338, is aligned with a second detector module electrical contact (e.g., 232). Further still, the third base electrical contact 344, which is located the furthest from the center of the module receptacle 338 relative to the other base electrical contacts, is aligned with a third detector module electrical contact (e.g., 234). Because they are aligned, once the detector module 304 is fully inserted into the module receptacle 338 of the base 302, the base electrical contacts 340, 342, 344 make electrical contact with the corresponding detector module electrical contacts, thereby putting the base electrical contacts 340, 342, 344 in electric communication with the detector module electrical contacts.

As described elsewhere herein, each of the base electrical contacts 340, 342, 344 can be in communication with electronics within the base 302 while the detector module electrical contacts can be in communication with electronics within the detector module 304. Accordingly, once the detector module 304 is fully inserted into the base 302 and the detector module electrical contacts are in electrical communication with the base electrical contacts 340, 342, 344, the electronics of the detector module 304 can be in electrical communication with the electronics in the base 302.

The electrical communication between the detector module 304 and the base 302 via their electrical contacts can enable power and/or data to transfer between them. Regarding data transfer, in the illustrated embodiment, the hazard sensor 322 of the detector module 304 can detect a hazard and provide an output to the base 302 indicating that a hazard has been detected. In response, the base 302 can activate an audio and/or visual alarm to alert persons of the detected hazard. In some examples, the detector module 304 can receive one or more measurements taken by the optional sensor 352 and can use the one or more measurements to determine if a hazard is present, in addition to or in lieu of hazard sensor 322 measurements. Additionally or alternatively, in some examples, the detector module 304 can send one or more measurements taken by the hazard sensor 322 to the base 302.

Regarding power transfer, in some examples, the detector module 304 can receive power from the base 302 via the detector module electrical contacts and base electrical contacts 340, 342, 344. In some such examples, the detector module can receive primary power from the base with the detector module's battery (e.g., 220) providing backup power to the detector module in case the primary power from the base is unavailable. Alternatively, in some examples, the detector module receives primary power from its battery (e.g., 220) and can receive backup power from the base in case the primary power from the battery is unavailable.

While the illustrated embodiment of FIG. 3 has three base electrical contacts 340, 342, 344 and three detector module electrical contacts, any number of base electrical contacts and detector module electrical contacts are contemplated. For instance, in some examples, the base includes two base electrical contacts while the detector module includes two detector module electrical contacts. In most embodiments, the number of base electrical contacts matches the number of detector module electrical contacts.

Additionally, while the base electrical contacts 340, 342, 344 comprise protruding, spring-loaded contacts and the detector module electrical contacts comprise substantially flat, concentric circle contacts, in some examples, the types of electrical contacts are switched. For example, the base electrical contacts can comprise substantially flat, concentric circle contacts while the detector module electrical contacts comprise protruding, spring-loaded contacts. However, both such configurations have an advantage of maintaining the base in electrical communication with the detector module no matter the rotational orientation of the detector module relative to the base. For example, the detector module can be fully inserted at 0 degrees, 90 degrees, 180 degrees, 270 degrees, or any other amount relative to the base and the detector module electrical contacts will be in electrical communication with the base electrical contacts. Further, rotating the detector module relative to the base when the detector module is fully inserted will not cause the detector module to break electrical communication with the base.

Continuing with FIG. 3, the base 302 includes the switch 346 located within the module receptacle 338. The switch 346 can interface with electronics in the base 302 such as audio and/or visual alarms (e.g., lights, speakers). In FIG. 3, the switch is in the form of a button. To activate the switch 346, the detector module 304, after being fully inserted, can be pressed further into the module receptacle 338. A rear flat portion of the detector module 304 can thus press into the switch and cause it to activate. The base electrical contacts 340, 342, 344 in FIG. 3 are spring loaded and can compress, thereby enabling the detector module 304 to be pressed further into the module receptacle 338 after full insertion of the detector module into the module receptacle. Such a configuration can prevent permanent deformation of the contacts and prevent breaking electrical contact between the base 302 and the detector module.

In some examples, activation of the switch can cause one or both of the base 302 or the detector module 304 to perform a function. For instance, in one such example, when the switch is activated (e.g., pressed), the detector module can perform a self-test function to assess operation of the detector module and communicate a result of the self-test to an external device (e.g., 250) via communication circuitry (e.g., 226). A self-test function can test if the detector module is operating properly, such as having a properly functioning battery and hazard sensor. In other examples, with the detector module 304 fully inserted into the module receptacle 338, activation of the switch can cause the base 302 to activate or deactivate an audio and/or visual alarm contained within the base. For instance, a user can press the detector module 304 into the base 302 to cause an audio and/or visual alarm to activate, thereby testing to see if the base 302 is operating properly. Additionally or alternatively, in an instance where the base is already in an alarm state (e.g., audio and/or visual alarms being active), a user can press the detector module 304 into the base 302 to cause an audio and/or visual alarm to deactivate. A person having ordinary skill in the art will appreciate that activation of the switch can be used to “test” the hazard detector device and/or “silence” the hazard detector device as is commonly understood with respect to smoke detectors and other common hazard detectors.

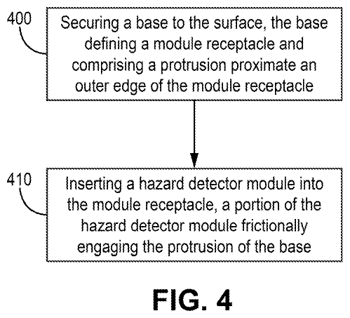

Moving to FIG. 4, FIG. 4 is a flowchart of an example method of installing a hazard detector device comprising a base and a hazard detector module according to an aspect of the present disclosure. Starting with step 400, a user can secure the base to a surface such as a ceiling or wall. The base can define a module receptacle and include a protrusion located proximate an outer edge of the module receptacle. Next, a user can insert a hazard detector module into the module receptacle as in step 410. When inserting the hazard detector module, a portion of the hazard detector module can frictionally engage the protrusion of the base. The frictional engagement can ensure the hazard detector module is secured to the base. In the example method, the hazard detector module includes a battery, a hazard sensor powered by the battery, a memory configured to store data about the hazard sensor, and communication circuitry in communication with the memory configured to communicate with an external device. As described elsewhere herein, the hazard sensor can comprise a smoke sensor.

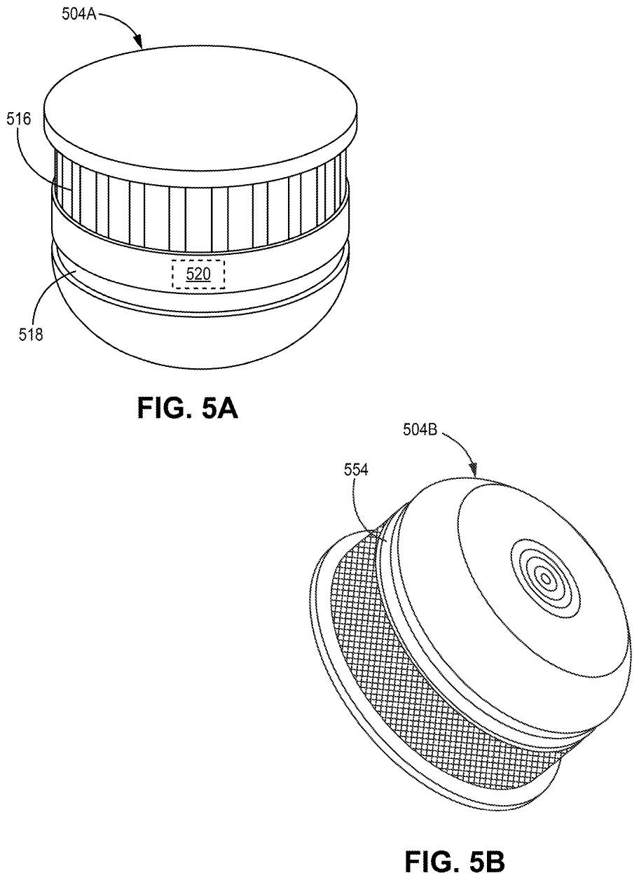

Moving to FIG. 5A and FIG. 5B, FIG. 5A and FIG. 5B are perspective views of alternate examples of detector modules according to aspects of the present disclosure. In comparison to the detector module 204 of FIG. 2A, the detector module 504A of FIG. 5A includes many of the same structures but has a different grill 516. The grill 516 is not in the form of a mesh of small holes, but instead includes a series of larger vertical openings which allow gases, particulates, and heat to reach a hazard sensor located inside the detector module while preventing external debris from reaching the hazard sensor. Additionally, the detector module 504A includes a battery 520 which has a smaller capacity than the battery 220 of FIG. 2A. The smaller capacity battery 520 can reduce the cost of the detector module 504A.

In comparison to the detector module 204 of FIG. 2B and the detector module 504A of FIG. 5A, the detector module 504B of FIG. 5B does not include an indent (e.g., 218, 518). Instead, the detector module 504B includes a lip 554 that surrounds the detector module 504B. The lip 554 can be used in securing the detector module 504B to a module receptacle. In some examples, the lip 554 prevents the detector module 504B from being inserted too far into a module receptacle. Additionally, the detector module 504A can comprise different materials than the detector module 204 or the detector module 504A. For instance, in FIG. 5B, the detector module 504B has a rear portion that comprises a more rubber-like material which can increase friction between the detector module and a module receptacle.

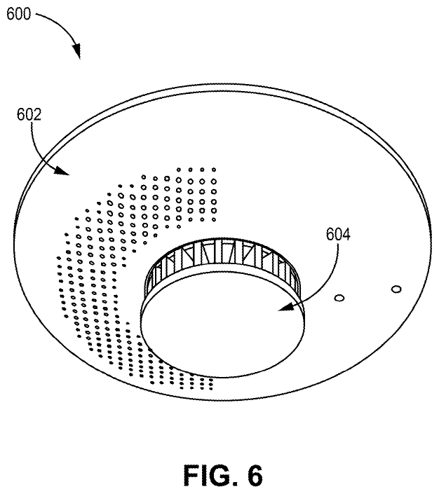

Moving to FIG. 6, FIG. 6 is a perspective view of an alternate example of a hazard detector device 600 including a base 602 and detector module 604 according to an aspect of the present disclosure. As illustrated, the base 602 and detector module 604 can have a different design when compared to the base and detector module of FIG. 1, for example.

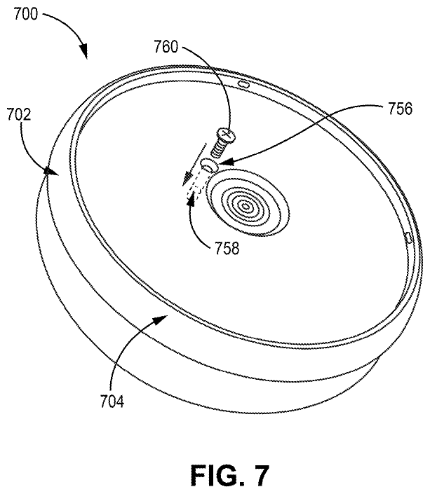

Moving to FIG. 7, FIG. 7 is a perspective view of an alternate example of a hazard detector device 700 including a base 702 and detector module 704 according to an aspect of the present disclosure. In the illustrated example, the base 702 defines a first security hole 756 which goes through the entire base 702 while the detector module 704 defines a second security hole 758 that is located wholly within the detector module 704. The first security hole 756 and the second security hole 758 are sized to accept a security fastener 760 such as a screw. In the illustrated embodiment, the first security hole 756 and the second security hole 758 are threaded as is the security fastener 760. The security fastener 760 can be inserted through the first security hole 756 of the base 702 and end within the second security hole 758 of the detector module 704. When inserted, the security fastener 760 can prevent the detector module 704 from being easily removed (e.g., simply pulled) from the base 702. This can be advantageous as it can prevent tampering with the hazard detector device 700. Other methods of securely mounting the detector module 704 in the base 702 are contemplated. In some examples, securely mounting the detector module to the base does not compromise the ability of the detector module to press into the base for testing and/or silencing operations as described elsewhere herein.

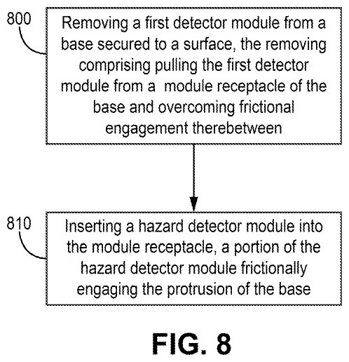

Moving to FIG. 8, FIG. 8 is a flowchart of an example method of replacing a detector module of an example hazard detector device according to an aspect of the present disclosure. The method starts at step 800 by removing a first detector module from a module receptacle of a base that is secured to a surface, with the removal comprising pulling the first detector module from the base and overcoming frictional engagement therebetween. In the method, the first detector module comprises a first battery, a first hazard sensor powered by the battery, a first memory configured to store data about the first hazard sensor, and first communication circuitry configured to communicate with an external device. The method continues at step 810 by installing a second detector module into the base. Installing the second detector module includes inserting the second detector module into the module receptacle of the base and frictionally engaging the second detector module with the module receptacle. The frictional engagement can in some examples, include overcoming friction between protrusions (e.g., 348) within the module receptacle and the detector module itself such that the protrusions engage an indent (e.g., 318) of the detector module.

In the example method of FIG. 8, the second detector module can comprise a second battery, a second hazard sensor powered by the second battery, a second memory configured to store data about the second hazard sensor, and second communication circuitry configured to communicate with the external device. In some examples, the second detector module can have more and/or better features than the first detector module. For instance, in some examples, the first battery has capacity to power the first hazard sensor for between one year and three years while the second battery has capacity to power the second hazard sensor for more than three years (e.g., 10 years). In such examples, the first battery can comprise an alkaline battery while the second battery can comprise a lithium battery. Additionally, in some examples, the second hazard sensor can be different from the first hazard sensor. For instance, the second hazard sensor can comprise a smoke sensor and a carbon monoxide sensor while the first hazard sensor comprises a smoke sensor but not a carbon monoxide sensor. Being able to exchange an existing detector module with a new detector module having more and/or better features can be advantageous, and users do not need to install another base to easily install a better detector module.

Various examples have been described. These and other examples are within the scope of the following claims.

What is claimed is:

1. A hazard detector device comprising:
a base defining a module receptacle and configured to be secured to a surface; and
a detector module configured to insert into the module receptacle and secure to the base, the detector module comprising:
a battery;
a hazard sensor powered by the battery;
a memory configured to store data about the hazard sensor; and
communication circuitry in communication with the memory and configured to communicate with an external device.

2. The hazard detector device of claim 1, wherein the hazard sensor comprises a smoke sensor.

3. The hazard detector device of claim 2, wherein the hazard sensor further comprises a carbon monoxide detector.

4. The hazard detector device of claim 1, wherein:
the base comprises one or more of a temperature sensor, a humidity sensor, a motion sensor, a pressure sensor, or a light sensor; and
the detector module is configured to receive one or more measurements from the one or more of the temperature sensor, the humidity sensor, the motion sensor, the pressure sensor, or the light sensor and use the one or more measurements with one or more measurements from the hazard sensor to determine if a hazard is present.

5. The hazard detector device of claim 1, wherein:
the base comprises one or more base electrical contacts and the detector module comprises one or more detector module electrical contacts; and
the one or more detector module electrical contacts is in electrical communication with the one or more base electrical contacts when the detector module is inserted into the base.

6. The hazard detector device of claim 5, wherein the one or more base electrical contacts comprise concentric circles of electrical contacts and the one or more detector module electrical contacts comprise spring-loaded electrical contacts.

7. The hazard detector device of claim 1, wherein:
the base comprises a switch;
the detector module activates the switch of the base when the detector module is pressed into the base; and
the detector module, when pressed into the base. is configured to:
perform a self-test to assess operation of the detector module; and
communicate a result of the self-test to the external device via the communication circuitry.

8. The hazard detector device of claim 7, wherein the base comprises an audio and/or visual alarm configured to produce an alert when activated by the detector module during a hazard event, the detector module being fully inserted into the module receptacle to activate the audio and/or visual alarm of the base, the audio and/or visual alarm configured to deactivate upon the detector module activating the switch of the base.

9. The hazard detector device of claim 1, wherein the memory is configured to store one or more items of information including one or more of: an installation date of the detector module when the detector module is first inserted into the base, a manufacturing date of the detector module, an expiration date of the detector module, a hazard sensor type, or a battery type.

10. The hazard detector device of claim 1, wherein the base provides primary power to the detector module via one or more electrical contacts and the battery provides backup power to the detector module.

11. The hazard detector device of claim 1, wherein:
the base comprises a protrusion extending at least partially into the module receptacle; and
the detector module, when inserted into the module receptacle, frictionally engages the protrusion to secure to the base.

12. The hazard detector device of claim 11, wherein:
the module receptacle and the detector module are generally cylindrical; and
the detector module comprises a ridge configured to contact the protrusion when the detector module is fully inserted into the module receptacle.

13. The hazard detector device of claim 1, wherein the detector module comprises a detecting portion, the detecting portion located outside the module receptacle when the detector module is secured to the base, the detecting portion configured to allow gases, heat, and particulates to reach the hazard sensor.

14. The hazard detector of claim 1, wherein the detector module defines a threaded hole, the threaded hole facing the base when the detector module is inserted into the module receptacle, the threaded hole configured to accept a fastener to secure the detector module to the base.

15. A method comprising:
securing a base to a surface, the base defining a module receptacle and comprising a protrusion proximate an outer edge of the module receptacle;
inserting a hazard detector module into the module receptacle, a portion of the hazard detector module frictionally engaging the protrusion of the base, the hazard detector module comprising:
a battery;
a hazard sensor powered by the battery;
a memory configured to store data about the hazard sensor; and
communication circuitry in communication with the memory and configured to communicate with an external device.

16. The method of claim 15, wherein the surface is a ceiling, and the hazard sensor is a smoke sensor.

17. A method comprising:
removing a first detector module from a base secured to a surface, the removing comprising pulling the first detector module from a module receptacle of the base and overcoming frictional engagement therebetween, the first detector module comprising:
a first battery;
a first hazard sensor powered by the first battery;
a first memory configured to store data about the first hazard sensor;
first communication circuitry configured to communicate with an external device; and
installing a second detector module into the base, the installing comprising inserting the second detector module into the module receptacle of the base and frictionally engaging the second detector module with the module receptacle.

18. The method of claim 17, wherein:
the second detector module comprises:
a second battery;
a second hazard sensor powered by the second battery;
a second memory configured to store data about the second hazard sensor; and
second communication circuitry configured to communicate with the external device;
the first battery has capacity to power the first hazard sensor for between one year and three years; and
the second battery has capacity to power the second hazard sensor for more than three years.

19. The method of claim 18, wherein the second hazard sensor comprises a smoke sensor and a carbon monoxide sensor, and the first hazard sensor comprises a smoke sensor but not a carbon monoxide sensor.

20. The method of claim 18, wherein the first battery comprises an alkaline battery and the second battery comprises a lithium battery.

21. The method of claim 17, wherein the second detector module includes a replacement date on a front face of the second detector module, the front face facing away from the surface when the second detector module is installed.

22. The method of claim 17, wherein the first detector module, the second detector module, and the module receptacle of the base are round.
